(19) United States
(12) Patent Application Publication
(10) Pub. No.: US 2014/0074231 A1
(43) Pub. Date: Mar. 13, 2014

(54) BIOCOMPATIBLE COMPOSITIONS AND METHODS OF MANUFACTURE

(71) Applicants: Michael Dominick DiVito, Minneapolis, MN (US); Allison Hubel, St. Paul, MN (US)

(72) Inventors: Allison Hubel, St. Paul, MN (US); Michael Dominick DiVito, Minneapolis, MN (US)

(73) Assignee: REGENTS OF THE UNIVERSITY OF MINNESOTA, St. Paul, MN (US)

(21) Appl. No.: 14/021,580

(22) Filed: Sep. 9, 2013

Related U.S. Application Data
(60) Provisional application No. 61/698,235, filed on Sep. 7, 2012.

(51) Int. Cl. A61L 31/12; A61F 2/14

(52) U.S. Cl. CPC A61L 31/125; A61F 2/142
USPC 623/5.11; 514/773

(57) ABSTRACT

This disclosure describes, in one aspect, a method that generally includes combining a silica precursor and a biocompatible polymer under conditions effective for the silica precursor and the biocompatible polymer to form a gel, at least partially dehydrating the gel, and rehydrating the gel. This disclosure also describes a corneal implant prepared by any embodiment of the general method described herein.

### CROSS-REFERENCE TO RELATED APPLICATION

This application claims priority to U.S. Provisional Patent Application Ser. No. 61/698,235, filed Sep. 7, 2012, which is incorporated herein by reference.

### SUMMARY

This disclosure describes, in one aspect, a method that generally includes combining a silica precursor and a biocompatible polymer under conditions effective for the silica precursor and the biocompatible polymer to form a gel, at least partially dehydrating the gel, and rehydrating the gel.

In some embodiments, the biocompatible polymer comprises Type I collagen. In some embodiments, the silica precursor and the biocompatible polymer are provided in amounts to produce a gel comprising a silica:biopolymer ratio of no more than 9:1. In some of these embodiments, the silica:biopolymer ratio is no more than 3:1.

In some embodiments, dehydrating the gel can include incubating the gel in relative humidity of no more than 75%.

In some embodiments, dehydrating the gel can include incubating the gel at a temperature of from 15° C. to 40° C.

In some embodiments, dehydrating the gel can include incubating the gel for at least 24 hours.

In some embodiments rehydrating the gel can include incubating the at least partially dehydrated gel in a buffer.

In some embodiments, the silica precursor and the biocompatible polymer are combined in a solution having a pH of no less than 5.

In some embodiments, the silica precursor and the biocompatible polymer are combined in a solution having a pH of no more than 10.

In another aspect, this disclosure describes a method that generally includes combining a silica precursor, a biopolymer, and aqueous nano-crystalline cellulose under conditions effective for the silica precursor and the biopolymer to form a gel.

In another aspect, this disclosure describes a method that generally includes combining a silica precursor and a biopolymer solution under conditions effective for the silica precursor and the biopolymer in the solution to form a gel, wherein the biopolymer solution comprises the biopolymer at a concentration of at least 5 mg/mL.

In another aspect, this disclosure describes a corneal implant prepared by any of the methods summarized above.

The above summary is not intended to describe each disclosed embodiment or every implementation of the composites and/or methods described herein. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1. Anterior segment organ culture system. (left) Schematic illustration and (right) photograph of modified Petri dish with mounted anterior segment. Culture medium can be perfused into the artificial anterior chamber for medium exchange, and medium was filled external to the anterior segment to just cover the vertex of the cornea (dashed line).

FIG. 2. Assessment of closure of corneal epithelial defect. (left) The epithelial defect created by central lamellar keratectomy stained with sodium fluorescein. The size of the epithelial defect was determined daily until closure. (right) The same cornea after closure of the epithelial defect, devoid of central staining.

FIG. 3. Cross-sectional SEM of a 9:1 hydrogel at (A) lower magnification and (B) higher magnification.

FIG. 4. Optical characterization of the composite material: (A) 5 mm buttons submerged in PBS on top of a ruler. The 3:1 hydrogel button (7) was more transparent than the 3:1 xerogel button (8). (B) Light transmission curves in the visible range for 3:1 and 9:1 compositions in the hydrogel and xerogel states. The human cornea curve was derived from experimental data by van den Berg (van den Berg and Tan. Vision Res. 1994; 34(11):1453-1456). All of these curves were adjusted to a common thickness of 0.3 mm.

FIG. 5. Force-displacement curve for the 3:1 rehydrated xerogel from a suture pullout test.

FIG. 6. Light microscopy images of implanted rabbit cornea. The peripheral implant (arrows) was enveloped in a paracentral lamellar pocket of rabbit stroma, and the center of the implant was exposed in the region of the anterior lamellar keratectomy (denoted by asterisks). A thin layer of epithelium covered the implant and was continuous with the epithelium at the edge of the keratectomy. Inset, Higher magnification image of the area denoted by the box showing an intact thin layer of epithelium covering a thin implant. Hematoxylin and Eosin.

FIG. 7. High magnification of regenerated epithelium. (left) Stratified rabbit corneal epithelium attached to the acellular and amorphous implant. (right) Normal rabbit anterior cornea with stratified epithelium attached to cellular stroma. Hematoxylin and Eosin.

FIG. 8. SEM images of silica-composite composites of varying composition: (A) 9:1 hydrogel, (B) 9:1 xerogel, and (C) 3:1 xerogel. Silica aggregates (ellipsoid and spherical objects microns in size) can be seen in (A) and (C). Average fibril diameters with associate standard deviation are listed in the bottom left corner of each image.

FIG. 9. Histograms (A and C) of fibril diameters and corresponding the SEM images (B and D) of a 150 mg/mL collagen hydrogel with (A and B) and without (C and D) NCC16. There is a significant increase in fibril diameters upon addition of NCC16.

FIG. 10. SEM images at 10,000× of 5 mg/mL collagen hydrogel (A) and xerogel composite (B). Scale bars are 1 μm.

FIG. 11. Spectral transmittance of copolymerized silica-collagen composites in comparison to rabbit cornea data.

FIG. 12. Brightness values of backscatter/reflectance at different depths of collagen hydrogels measured with a clinical confocal microscope (A). Brightness values of theses samples at an arbitrary depth (B).

FIG. 13. Tensile properties of copolymerized composites (A) and a plot of these properties as a function of aging time for a 9:1 xerogel (B).

FIG. 14. Change in tensile properties of collagen hydrogels as concentration is increased via dialysis.

FIG. 15. Increase in mechanical properties of collagen hydrogel after silica addition via silica sol soaking Average mechanical properties for both hydrogels are listed, where the interval presented is the standard error.

FIG. 16. TEM images of the basal epithelial cell membrane interaction with 9:1 hydrogel implant (A), and control stroma (B). There is an interaction between the cell membrane and implant material, but no basement membrane that is indicated in the control by white arrows is apparent in the hydrogel implantation.

FIG. 17. Live (large cells with distinct morphology)/dead (small, round cells) fluorescent images for HFF cells attached to 9:1 hydrogel surface at (A) one hour, (B) 24 hours, and (C) 72 hours after a 1 hour incubation.

### DETAILED DESCRIPTION OF ILLUSTRATIVE EMBODIMENTS

This disclosure describes the manufacture and basic characteristics of a novel artificial corneal substrate that may promote corneal regeneration. In vitro and ex vivo studies indicate that the material has favorable optical properties and biocompatibility for use as an artificial cornea.

The cornea is a commonly transplanted tissue in the United States. Successful corneal transplantation can depend on the availability of good quality cadaveric donor tissue. Worldwide, the supply of donor corneas suitable for transplantation is insufficient to meet demand, partly because of a lack of appropriate eye banking facilities. Even in the United States, several factors impact the future efficient provision of donor tissue and the need for increasing the donor supply. Threats to the donor supply include, for example, the prevalence of common viral infections (e.g., hepatitis B), increasingly stringent FDA regulations towards testing for emerging infections (EBAA. Eye Bank Association of America. Annual statistical report. 2010), and the large number of potential donors who have had cataract or refractive surgery, which can render tissue ineligible for donation (Beck et al. Cornea. 2000; 19(3):503-510). In addition, the demand for donor corneas has increased because of an expanding population over age 65 years, and because improvements in surgical techniques have enabled safer and earlier intervention (Tan and Mehta. Cornea. 2007; 26:S21-S28).

Conventional artificial corneas (keratoprostheses) typically do not truly bio-integrate with surrounding tissues, nor do they typically allow for epithelialization of their surface, which can result in complications (Hicks et al. Eye. 2003; 17(3):385-392; Ma et al. Int. Ophth. Clinics. 2005; 45(4):49-59). As a result, it may be desirable to develop a biocompatible corneal substitute that can support tissue regeneration (Fagerholm et al. Sci. Transl. Med. 2010; 2(46):ra61). Regeneration of the cornea can involve materials that can mimic the optical and mechanical properties of the native cornea. In some cases, the materials also can be easily stored for use as needed, effectively reducing—even eliminating—the need for human donor tissue. Type I collagen-based structures have potential to meet these design criteria with biomechanical strength obtained by chemical cross-linking (Orwin et al. J. Biomech. Eng. 2003; 125(4):439; Crabb and Hubel. Tissue Eng. Part A. 2008; 14(1):173-82; Duncan et al. Biomaterials. 2010; 31(34):8996-9005; Liu et al. Invest. Ophth. & Visual Sci. 2006; 47(5):1869-1875). Cross-linked collagen constructs have enabled tissue regeneration, and have been piloted in human trials in vivo with moderate but incomplete success (Fagerholm et al. Sci. Transl. Med. 2010; 2(46):ra61; Crabb et al. Tissue Eng. 2006; 12(6):1565-1575; Fagerholm et al. Clin. Transl. Sci. 2009; 2(2):162-164).

As an alternative to cross-linked collagen constructs, we have developed alternative compositions of silica/biocompatible polymer composite materials. We show that, for example, silica precursor 3-aminopropyltriethoxsilane (APTS) and, for example, soluble type I bovine collagen can be combined to create a bio-composite gel. We determined the mechanical and optical properties for different silica/collagen compositions in hydrogel and xerogel states, and we developed a manufacturing process to construct implants of the appropriate size and geometry. We report the microstructure, biomechanical, and optical properties of these implants, and biocompatibility studies in a cultured rabbit cornea model.

A silica/biopolymer composite can be manufactured in a manner to permit bottom-up organization of the matrix. Specifically, for example, one can control major microstructural characteristics (e.g., fibril diameter and/or fibril density) in order to achieve macroscopic properties (e.g., optical and/or biomechanical properties) of interest. We describe herein different techniques developed to achieve microstructural control.

While certain exemplary embodiments are described below in the context of a silica/biopolymer composite in which the biopolymer includes collagen (i.e., a silica/collagen composite), the composite and methods described herein can include and/or can involve using other biopolymers as described in more detail below. Thus, wherever it may be applicable, reference to a silica/collagen composite applies equally to a silica/biopolymer composite that includes any other suitable biopolymer.

In some embodiments, the silica/biopolymer composite can be prepared using a copolymerization process. Soluble type I bovine collagen (5 mg/mL; Organogenesis; Canton, Mass.) was mixed with a pre-hydrolyzed solution 3-aminopropyltriethoxysilane (APTES; Sigma-Aldrich; St. Louis, Mo.) to neutral a pH, causing simultaneous collagen fibril formation and silica polymerization.

The composite may be prepared using any suitable ratio of silica-to-biopolymer, as described in more detail below. The description that follows refers to exemplary embodiments that possess a silica-to-collagen weight ratio of either 3:1 or 9:1. Other embodiments can include similar ratios using other biopolymers, as described immediately above, or other silica-to-biopolymer weight ratios, as described in more detail below.

### Microstructural Properties

Fibril Diameter

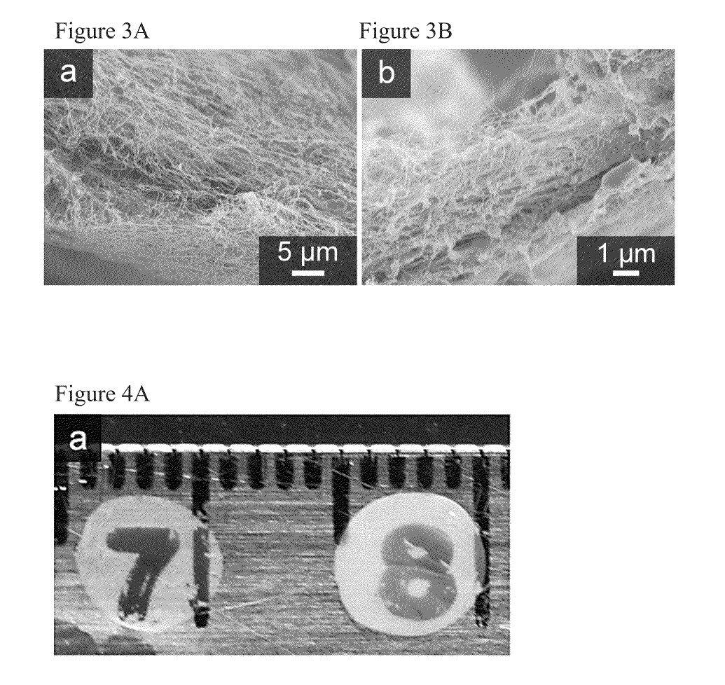

Scanning electron microscopy (SEM) images presented in FIG. 3 show that the 9:1 hydrogel material consisted of a network of evenly sized fibers (FIG. 3a). Non-fibrillar structures on the surface of the fibrillar network appear to be silica aggregates (FIG. 3b).

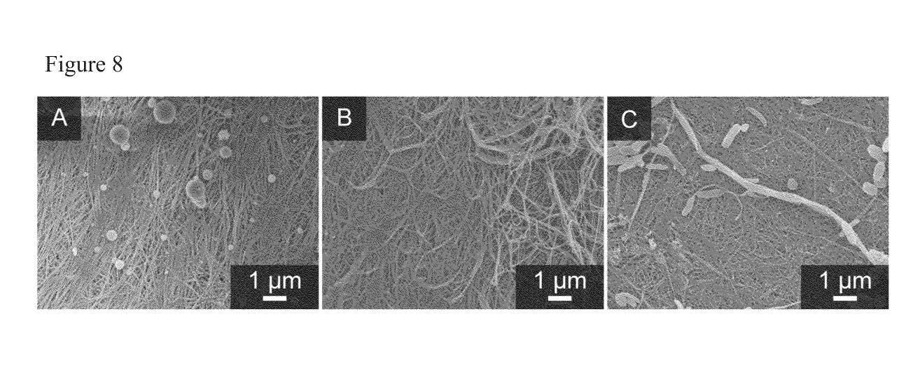

Two hydrogel compositions of different silica precursor-to-collagen weight ratios were prepared (3:1, 9:1). FIG. 8 shows SEM images that show an increase in average fibril diameter with a greater silica precursor weight percentage (compare FIG. 8B and FIG. 8C).

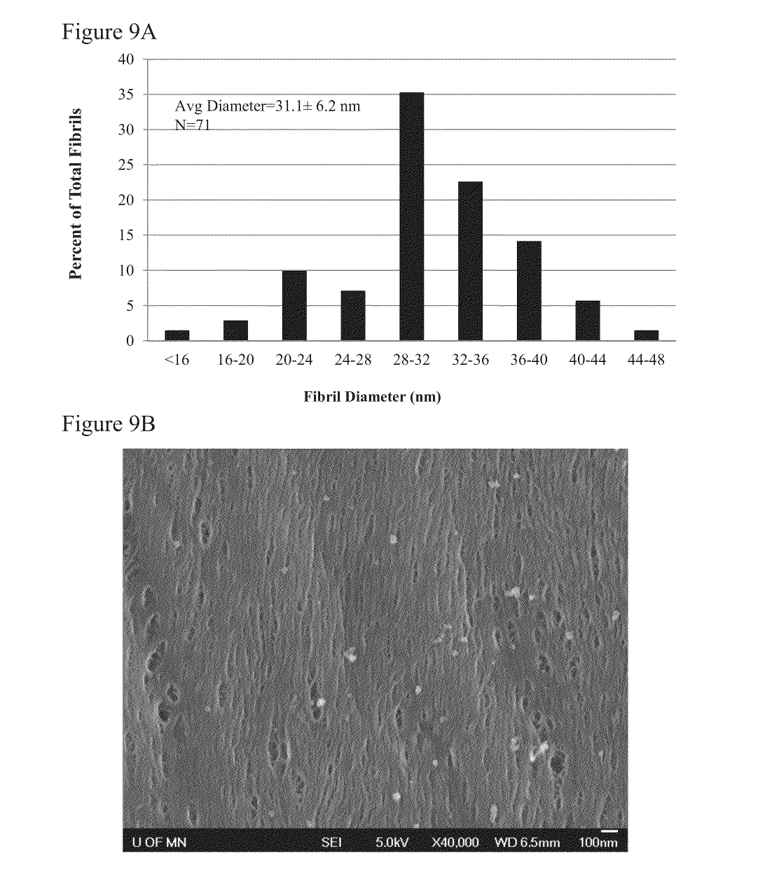

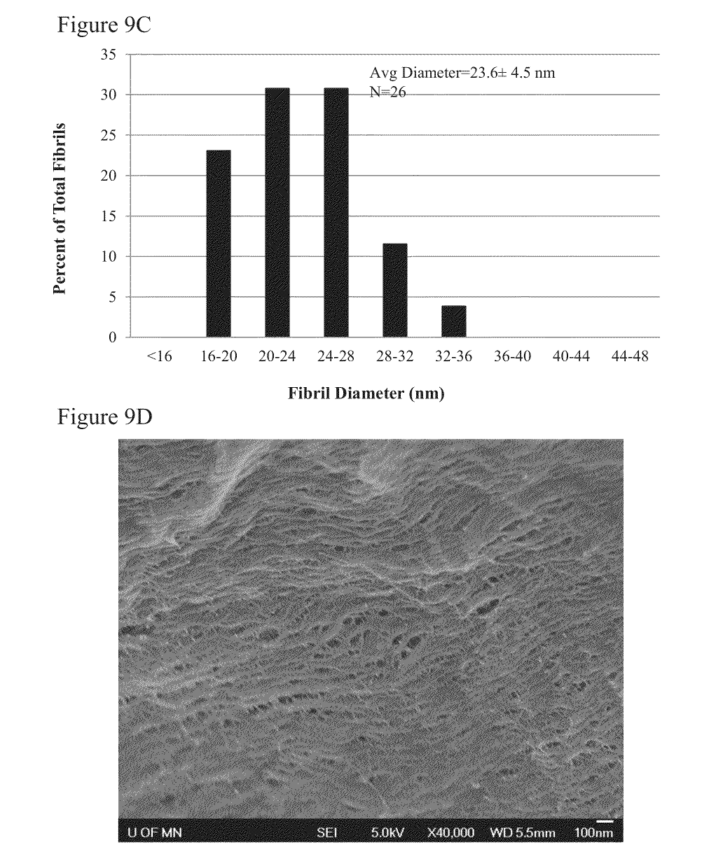

Mixtures of aqueous nano-crystalline cellulose (NCC) suspensions and soluble type I rat tail collagen (Olaf; Worcester, Mass.) were exposed to ammonia vapors, causing collagen fibrils to form in the presence of NCC particles. FIG. 9 presents data showing that the average fibril diameter increases with the addition of NCC (compare FIGS. 9A and B (with NCC) with FIGS. 9C and D (without NCC)). Moreover, the addition of NCC also can reduce the dispersity of collagen fibril diameters to a smaller range.

Fibril Spacing

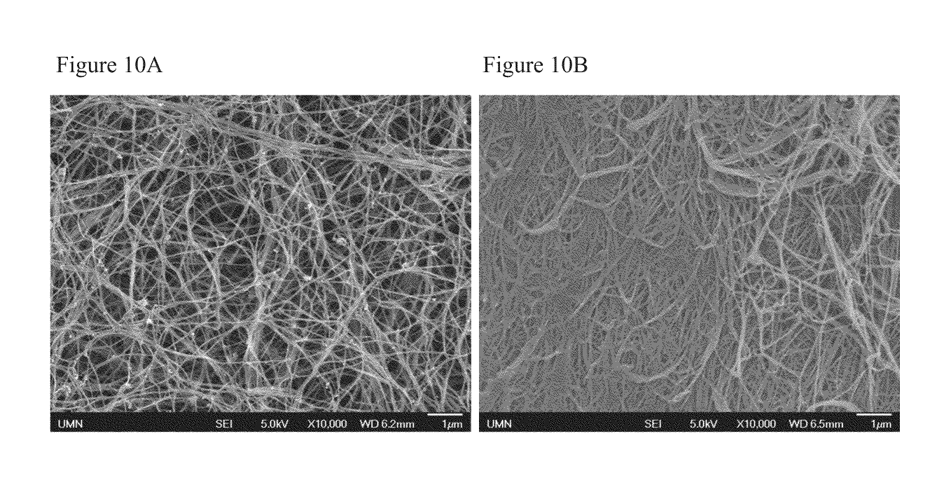

The density of fibrils, or average spacing, can be modulated to control mechanical properties. The first approach taken was to air dry and rehydrate hydrogels to create xerogels of greater fibril densities. It is difficult to characterize the spacing with SEM, but the decrease in fibril spacing of xerogels with respect its original hydrogel state is apparent from just a qualitative inspection (FIG. 10). The dense networks of fibrils created by this method resulted in a decrease in optical quality.

Our second approach involved increasing the collagen concentration of the initial collagen solutions prior to fibril formation. This approach involved dialysis to increase the concentration of liquid collagen solutions as high as 150 mg/mL. Briefly, the stock rat tail collagen solution was placed in cellulose dialysis tubing and dialyzed against a (poly)ethylene glycol solution. Target concentrations were met by periodically checking the weight of the collagen solution throughout dialysis. The hydrogels in FIG. 9 were concentrated to 150 mg/mL using this method, and significant decreases in fibril spacing and increases in organization can be observed.

### Optical Properties

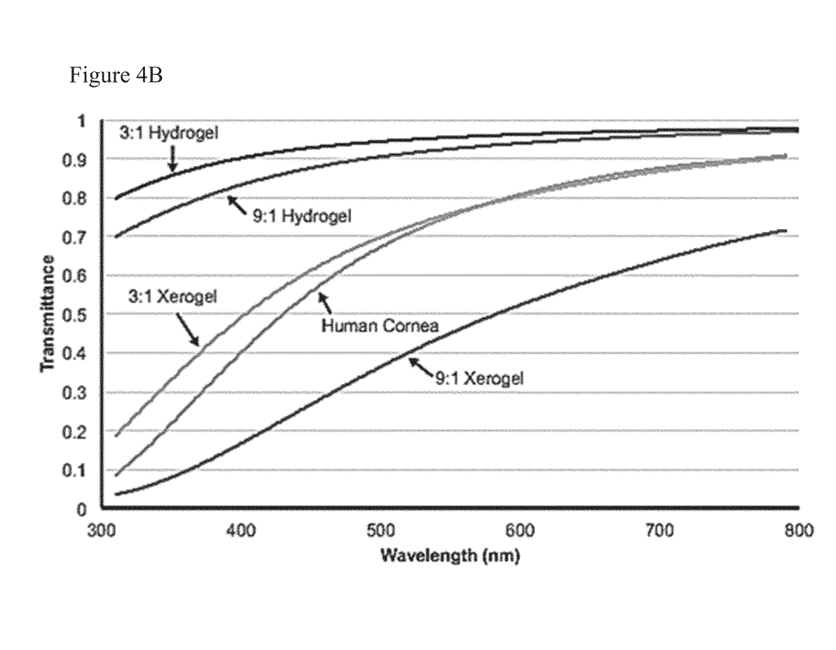

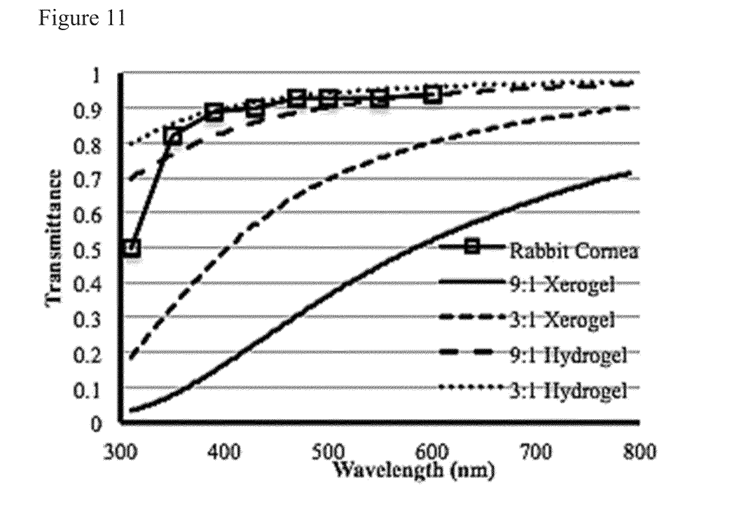

Transmittance increased from the ultraviolet to the infrared range for both compositions (9:1 silica:collagen vs. 3:1 silica:collagen) and hydration states (hydrogel vs. xerogel). In general, the hydrogels had a higher transmittance than the xerogels (FIG. 4A and FIG. 11), and the lower silica composite had a higher transmittance than the higher silica composite. The transmittance-wavelength curves for all composites and hydration states are shown in FIG. 4B and FIG. 11. The value of transmittance for the human cornea curve was adapted from an experimental transmittance expression found by van den Berg, and corneal thickness measurements published by Muscat (van den Berg and Tan. Vision Res. 1994; 34(11):1453-1456; Muscat et al. Invest. Ophth. & Visual Sci. 2002; 43(6):1791-5). Equation (1) was used to adjust each curve to a common thickness of 0.3 mm. The 3:1 hydrogel data also was consistent with data previously published by McLaren et al. for rabbit corneas (FIG. 11). Both xerogel composites had lower transmittance than the hydrogel composites over the entire UV/Visible light spectrum investigated (FIG. 11).

Additionally, an Abbe refractometer was used to measure the macroscopic indices of refraction for the same samples. Composites with higher silica concentrations exhibited higher indices of refraction, which are close to values reported for bulk amorphous silica. Moreover, all samples exhibited refractive indices close to those reported for the human cornea. (Table 1).

TABLE 1
Indices of refraction for copolymerized
composites compared to human cornea

| Material | Index of Refraction |
|---|---|
| Human cornea | 1.37-1.40 |
| 3:1 Hydrogel | 1.332 |
| 3:1 Xerogel | 1.389 |
| 9:1 Hydrogel | 1.334 |
| 9:1 Xerogel | 1.403 |

The refractive index was 1.334 and 1.332 for the 9:1 and 3:1 hydrogel compositions, respectively. Creation of a xerogel increased the index of refraction to 1.403 and 1.389 respectively.

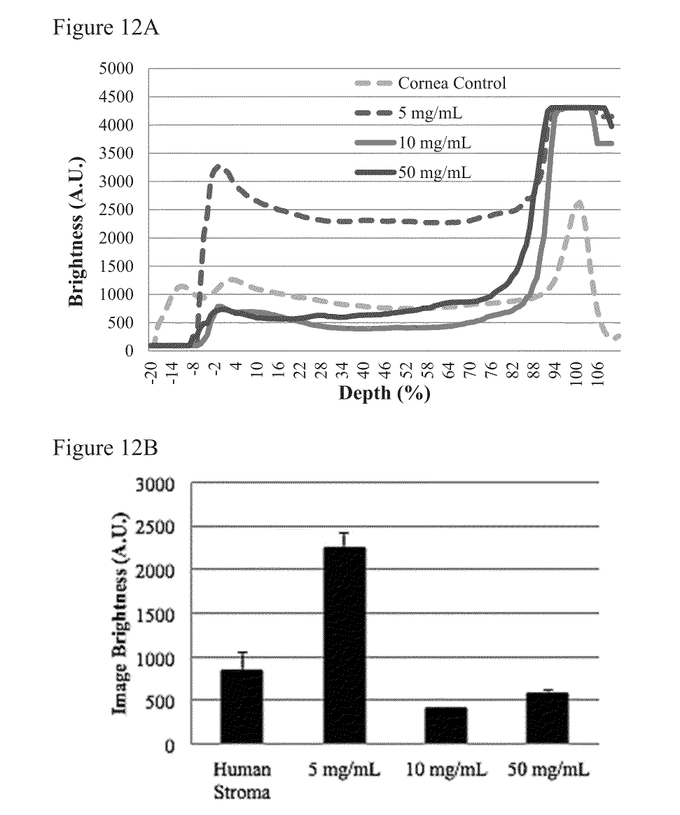

To compare the light scattering properties of collagen hydrogels made from different initial collagen concentrations obtained using the dialysis techniques described above, backscatter of incident light on samples was measured using a clinical confocal microscope. Briefly, flat hydrogel samples of thicknesses similar to the native cornea were prepared and imaged in a vertically standing petri dish. A lamp of a known power applied incident light perpendicular to the sample surface, and the brightness of the reflected/backscattered light was measured at various focal planes throughout entire thickness of the sample using a confocal microscope. The brightness curves associated with the same incident light to varying concentrations of hydrogels is shown in FIG. 12. The 5 mg/mL collagen hydrogel scattered the most light, which was over double of what was reported for 36 healthy human corneas. However, the degree of backscatter was shown to decrease after higher concentrations were reached with dialysis methods, dipping below the human cornea control. These results agreed with quick qualitative assessments made of these gels, and validated the dialysis procedures used to achieve higher concentrations—i.e. lower fibril spacing and improved mechanical properties.

### Biomechanical Analysis

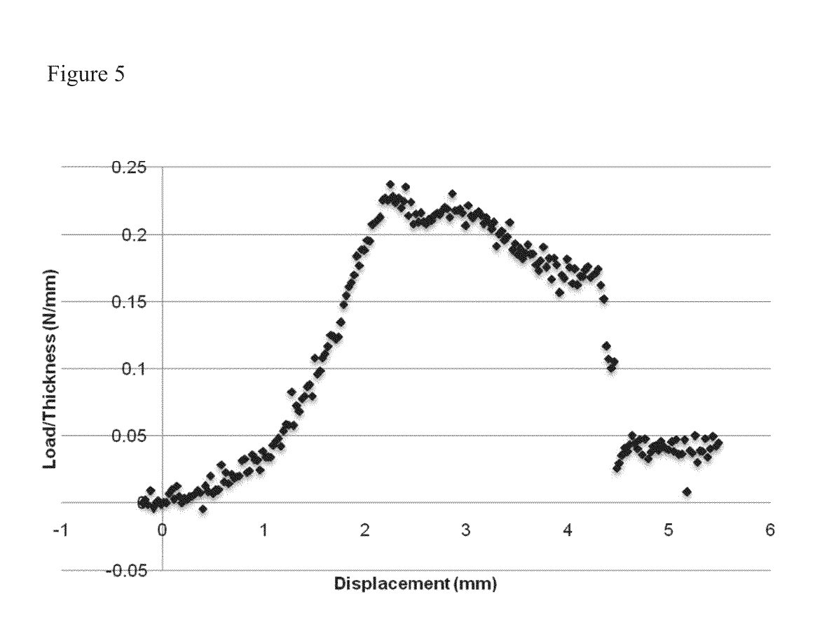

An example of a rupture strength-displacement curve for a 3:1 xerogel specimen is shown in FIG. 5. For these tests, an ophthalmological nylon suture loop was place through an 8 mm disc sample 2 mm perpendicularly inward from the edge. Using a low force biaxial tester, a constant displacement was applied to suture thread creating a force at the suture site. The force per unit thickness of the sample at failure was termed the suture pullout strength, and the average suture pullout strength of 3:1 xerogels is presented in FIG. 5. The material displayed strain-hardening behavior prior to failure, and then proceeded to rupture in a ductile manner. The rupture strength of the 3:1 xerogel was 0.161±0.047 N/mm.

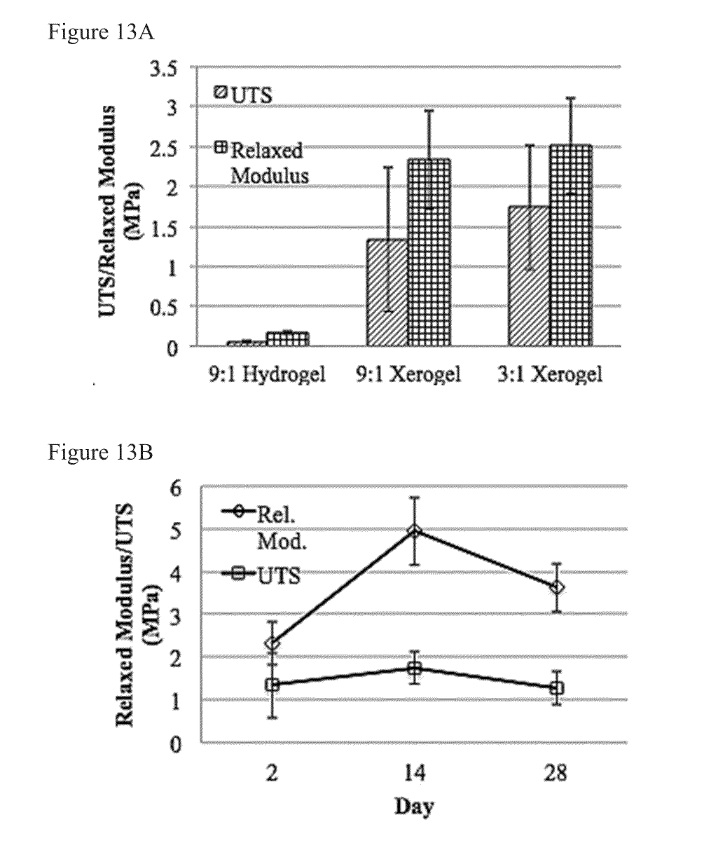

Uniaxial tensile tests were performed on the same set of materials with the same low force biaxial tester. FIG. 13 shows that the strength and stiffness increases by an order of magnitude going from a hydrogel to xerogel state. The 3:1 xerogels that displayed the best mechanical properties for suture pullout also had a higher ultimate tensile strength (UTS) and relaxed modulus than the 9:1 composition (FIG. 13A).

In addition to these tensile results, the variation of strength and stiffness of a silica/collagen composite with aging was measured. Tensile tests were performed on 9:1 xerogels that had been aged in a neutral aqueous solution for two days, 14 days, or 28 days. While UTS remained relatively constant, relaxed modulus increased in the first two weeks of aging, and then began to decrease in the last two weeks.

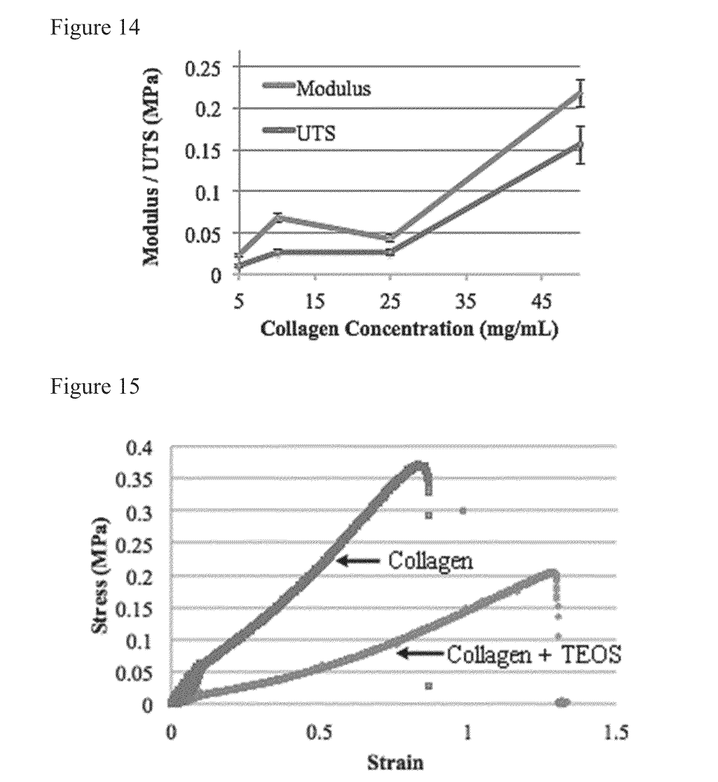

The mechanical behavior of concentrated gels made using the dialysis procedure described above, which displayed low light scattering behavior similar to the cornea, was characterized. FIG. 14 shows an order of magnitude increase in strength and stiffness when dialyzing from a collagen concentration of 5 mg/mL to a collagen concentration of 50 mg/mL. The collagen concentration of 50 mg/mL was chosen to be characterized because this is the approximate collagen concentration in xerogels produced from 5 mg/mL hydrogels. Pure collagen xerogels prepared from the same rat-tail collagen used for the dialysis samples had an average modulus of 0.195 and an average UTS of 0.077 MPa. Therefore, dialysis is a validated method of producing hydrogels with a similar modulus, higher UTS, and improved optical behavior when compared to xerogels of the same concentration.

In some embodiments, the method of preparing the silica/biopolymer composite can involve two separate processing steps: collagen gelation and silica deposition, e.g., soaking a collagen hydrogel in a silica sol at, for example, a neutral pH. The sol used was a mixture of distilled water, triethyloxysilane (TEOS), and ethanol at a volume ratio of 2:2:5, respectively. FIG. 15 shows that one can achieve increases in stiffness and strength of a 50 mg/mL collagen hydrogel by soaking the collagen hydrogel in a silica precursor for 18 hours. The control collagen material was soaked in the same molarity of ethanol as the composite material to correct for any changes associated with hydration of the collagen fibrils.

### Biocompatibility in a Rabbit Model

A rabbit corneal organ culture model was used to study the biocompatibility of re-epithelialization of 9:1 hydrogel implants. The hydrogel was copolymerized in a lens shaped mold and trephined to make 8 mm diameter button implants. The anterior portion of a rabbit eye was excised and placed in the modified organ culture apparatus shown in FIG. 1. The top 200 μm of the anterior stroma was excised, and a 9 mm diameter circular lamellar pocket was created at a depth of 200 μm from the anterior surface. The hydrogel implant was placed on the anterior surface, and the edges were inserted into the peripheral lamellar pocket.

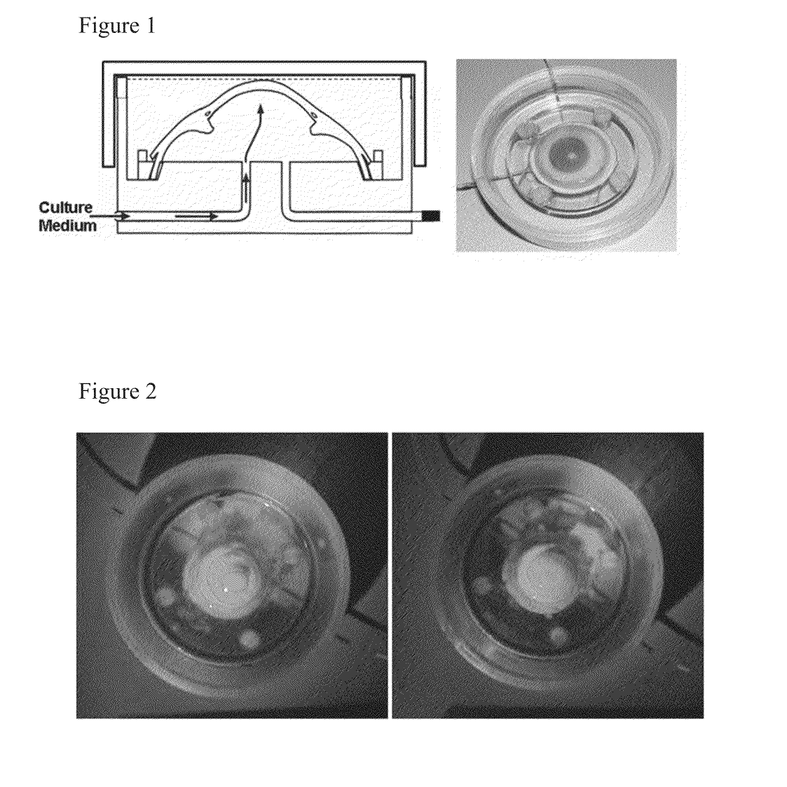

The epithelial defect was monitored every day by staining with sodium fluorescein and observing with a blue light (FIG. 2). Eight rabbit corneas received surgery with implantation of a composite implant, and two corneas received surgery without implantation (controls). In all corneas, epithelial defects immediately after surgery were approximately 5 to 6 mm in widest diameter (FIG. 2). In the two control corneas, complete re-epithelialization over exposed bare stroma took 8 and 15 days, respectively. In experimental corneas, two corneas became contaminated during the culture period and were excluded from analysis; the contamination was unrelated to sterility of the implants. In the remaining six implanted corneas, complete re-epithelialization over the exposed implant took 5.5±2.4 days (range, 3-10 days; FIG. 2; Table 2).

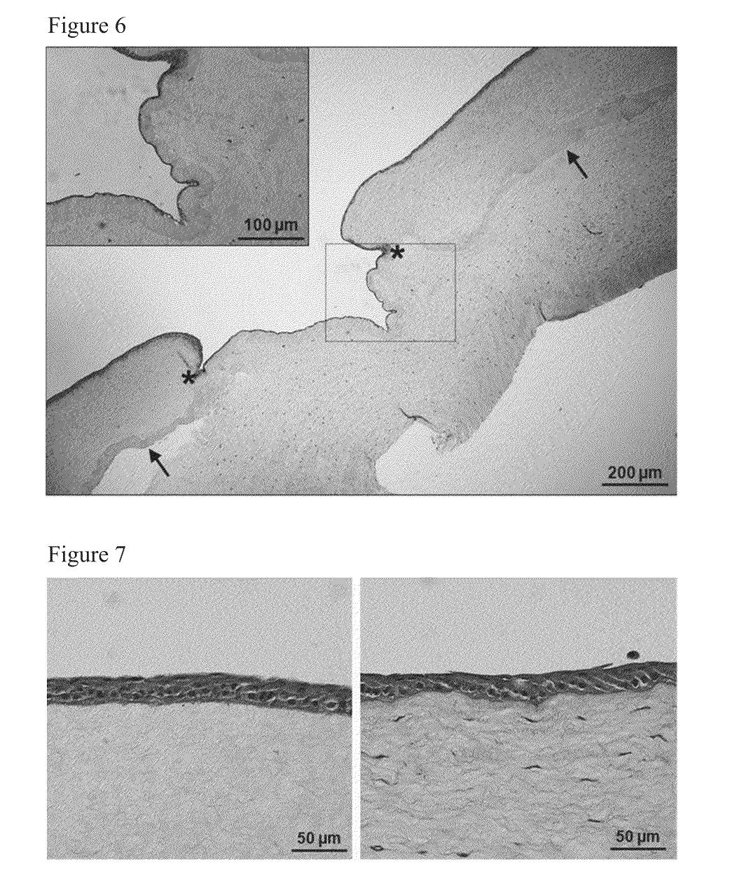

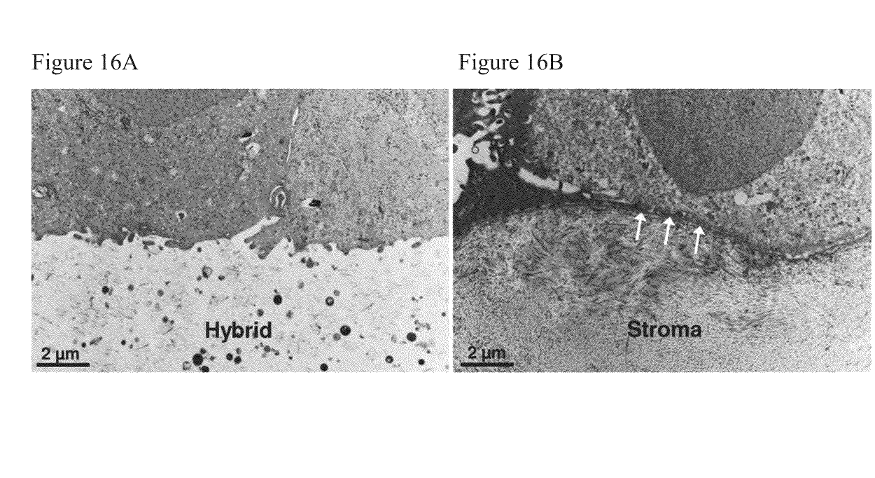

Epithelialization of the implant surface was confirmed by light microscopy in all cases (FIG. 6). An early stratified epithelium was noted with longer duration of culture, but there was no evidence of stromal cell migration into the implants at the end of the culture period (FIG. 7). Once the epithelial defect was completely closed, the samples remained in culture for additional days, and then histological sections were stained and imaged (FIG. 7). In general, the composite hydrogels showed good biocompatibility and were able to support epithelial growth on its surface and maintain a normal epithelium compared to the rabbit stroma control. The details of each sample are summarized in Table 2. TEM images were also obtained to show the hydrogel-basal membrane interaction (FIG. 16).

TABLE 2
Summary of ex vivo results of 9:1 hydrogel implants
compared to control rabbit corneas.

| | | | Histological Analysis | | |
|---|---|---|---|---|---|
| Epithelial Defect | | Time in | Layers of | | |
| | Time to | organ | epithelial | Keratin | Collagen |
| | closure | culture | cells (mean: | AE1/AE3 | IV |
| Size (mm) | (days) | (days) | [range]) | staining | staining |

Implanted Corneas

| | | | | | | |
|---|---|---|---|---|---|---|
| 1 | 5.5 × 4 | 6 | 9 | 1 [1-2] | + | − |
| 2 | 5 × 4 | 5 | 10 | 2 [1-3] | + | − |
| 3 | 5 × 5 | 10 | 10 | 1 [1-2] | + | − |
| 4 | 5 × 5 | 5 | 7 | 3 [1-5] | + | − |
| 5 | 5 × 5 | 3 | 7 | 3 [2-6] | + | − |
| 6 | 5 × 6 | 4 | 7 | 5 [4-8] | + | − |

Controls

| | | | | | | |
|---|---|---|---|---|---|---|
| 1 | 7 × 7 | 15 | 17 | 3 [1-5] | + | − |
| 2 | 5 × 5 | 8 | 10 | 2 [1-4] | + | − |

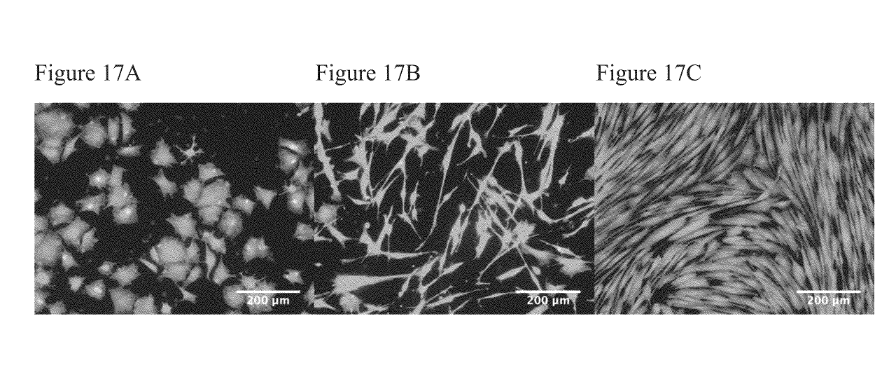

An in vitro cell-hydrogel culture method was developed to assess biocompatibility of the composites in a quick and easy fashion. Silica/collagen hydrogels were copolymerized in 6-well plates, and human foreskin fibroblast cells were suspended in a serum-free media. This cell solution was then pipetted on top of the hydrogel and incubated at 37° C. for one hour. The supernatant solution was then removed and replaced with normal cell culture media. A calcein AM/propidium iodide stain was then used to obtain live/dead images with a fluorescent light microscope at different time points. FIG. 17 shows that 9:1 hydrogels have sufficient biocompatibility to allow for fibroblast attachment after one hour, and a fully confluent layer of fibroblast after 72 hours. The dead cells present at time zero may be associated with detachment of cells during transport of cell culture to the imaging center.

In this study, we successfully engineered a model silica/biopolymer composite material as an artificial corneal stroma for implantation into an animal model. The material was transparent and could be manufactured to specific dimensions for surgical procedures. Basic biocompatibility was established by showing corneal re-epithelialization in an ex vivo rabbit model.

SEM images displayed the composite's fibrillar morphology. The absence of D-banding indicated that the collagen fibrils were coated with silica particles. The hydrogels had a higher transmittance than the native cornea. The 3:1 composition was stronger and more transparent than the higher silica composition. The index of refraction of the hydrogel samples were both approximately the same as that of water. After the samples were dehydrated, the refractive indices increased for composites of either silica:biopolymer ratio (3:1 or 9:1) compared to their respective hydrated hydrogels. This may be due, at least in part, to a decrease in the water content of the dehydrated composite and the index of refraction of silica being approximately 1.45 (Haynes, “Optical Properties of Selected Organic and Inorganic Materials.” CRC Handbook of Chemistry and Physics. 92nd ed. Boca Raton, Fla.: CRC Press/Taylor and Francis; 2012:163-4). Additionally, the higher silica content yielded a higher index of refraction, one closer to that of silica when compared to sample of lower silica content.

The epithelium of the human cornea has a refractive index of approximately 1.40, and the stroma ranges from 1.37 to 1.38 (Patel et al. J. Refractive Surg. 1995; 11(2):100-105). The 3:1 and 9:1 xerogel indices of 1.389 and 1.403, respectively, fall close to the human cornea values. The small difference between the 3:1 sample and the human stroma can be eliminated by minor adjustments in silica content.

The mechanical properties of the composite material are significantly improved when compared to a control made solely with collagen. In particular, hydrogels prepared with concentrated biopolymer solution (e.g., by dialysis prior to copolymerization) exhibited a similar modulus, higher UTS, and improved optical behavior when compared to xerogels of the same concentration.

The transmittance characteristics of the composite material have a distinct advantage over many other materials that have been investigated (FIG. 4; Liu et al. Biomaterials. 2009; 30(8):1551-9; Liu and Sheardown. Biomaterials. 2005; 26(3):233-44). The 3:1 xerogel mimics some measurements of native cornea closely in both the visible and UV spectrum. The 3:1 hydrogel has a transmittance spectrum in the visible that is similar to that of rabbit cornea in vivo (McLaren and Brubaker. Curr. Eye Res. 1996; 15(4):411-421; Walsh, et al. Physiol. Meas. 2008; 29 (2008):375-388), although it does not drop as quickly below 350 nm as it does in the living cornea. The high transmittance to 400 nm is consistent with measurements in human corneas where the corneas were not removed from the globe and were minimally disturbed (McLaren and Brubaker. Curr. Eye Res. 1996; 15(4):411-421).

For the initial biocompatibility studies, our goal was to show that corneal epithelium would regenerate across the surface of the implant. Without epithelium covering the implant surface, the implant can be prone to degradation and infection in vivo. We used an organ culture model to demonstrate that a surgically implanted bio-composite consistently supported complete re-epithelialization within an average of one week, similar to the healing time of a simple corneal epithelial defect of the same size. By histology, the corneal epithelium attached to the underlying composite. Moreover, the epithelium became stratified similar to normal rabbit epithelium with an extended period of culture. Epithelialization of any artificial corneal material can increase the clinical utility of the artificial material because the material is more likely to be retained. Our results represent an advantage compared to current plastic keratoprostheses, which do not support epithelium.

Thus, in one aspect, we describe a method that generally includes combining a silica precursor and a biocompatible polymer under conditions effective for the silica precursor and the biocompatible polymer to form a gel, at least partially dehydrating the gel, and rehydrating the gel.

Silica is naturally occurring and biocompatible. Silica gels, which can be produced by hydrolysis of a silica precursor, have been shown to be chemically inert, non-toxic, and can be engineered to have specific properties (porosity, optical clarity, mechanical stiffness, etc.). In addition, silica gels can be easily and cheaply manufactured into different shapes. The optical and manufacturing properties of silica gels are so favorable that microscope lens manufacturers have started to manufacture lenses from this material. Silica gels are also an excellent matrix for immobilization of macromolecules (proteins, drugs, etc.). Furthermore, silica influences self-assembly of collagen in vivo and provides potential as a template for controlling on a molecular level the nanostructure of the resulting matrix (Eglin et al., Biomed. Mater. Eng. 2005; 15:43-50).

Exemplary precursors for the silica component of the composite material include those shown in Table 3. The composite material can include any, or any combination of two or more, of the silica precursors listed in Table 3.

TABLE 3
Exemplary Silica Precursors

| Chemical Name | Acronym | Molecular Formula |
|---|---|---|
| Tetramethylorthosilicate | TMOS | Si(OCH₃)₄ |
| Tetraethylorthosilicate | TEOS | Si(OC₂H₅)₄ |
| Tetrakis(2-hydroxyethyl)orthosilicate | THEOS | Si(OCH₂CH₂OH)₄ |
| Methyldiethoxysilane | MDES | C₅H₁₄O₂Si |
| 3-(Glycidoxypropyl)triethoxysilane¹ | GPTES | C₁₂H₂₆O₅Si |
| 3-(Glycidoxypropyl)trimethoxysilane¹ | GPTMS | C₉H₂₀O₅Si |
| 3-(Trimethoxysilyl)propylacrylate | TMSPA | H₂C═CHCO₂(CH₂)₃Si(OCH₃)₃ |
| N-(3-Triethoxysilylpropyl)pyrrole | TESPP | |
| Vinyltriethoxysilane | VTES | H₂C═CHSi(OC₂H₅)₃ |
| Vinyltrimethoxysilane | VTMES | H₂C═CHSi(OCH₃)₃ |
| Methacryloxypropyltriethoxysilane | TESPM | |
| Silica Nanoparticles^{1, 2} | | SiO₂ |
| Sodium Silicate (e.g., 27% Silicic Acid 10% NaOH) | Water glass | |
| Diglycerylsilane¹ | DGS | |
| Methyltriethoxysilane¹ | MTMOS | CH₃Si(OCH₃)₃ |
| 3-aminopropyltriethoxysilane¹ | APTS | H₂N(CH₂)₃Si(OC₂H₅)₃ |
| 3-aminopropyltrimethoxysilane¹ | APTMS | C₆H₁₇NO₃Si |
| 3-(2,4-Dinitrophenylamino)propyltriethoxysilane | | |
| Mercaptopropyltriethoxysilane | TEPMS | HS(CH₂)₃Si(OCH₂CH₃)₃ |
| 3-(2-Aminoethylamino)propyltriethoxysilane | | (CH₃O)₃Si(CH₂)₃NHCH₂CH₂NH₂ |
| Isocyanatopropyltriethoxysilane¹ | | C₁₀H₂₁NO₄Si |
| Hydroxyl-terminated polydimethylsiloxane¹ | PDMS | |
| Triethoxysilyl-terminated polydimethylsiloxane¹ | PDMS | |
| Methyltriethoxysilane¹ | MTES | CH₃Si(OC₂H₅)₃ |

¹Exemplary precursors that may function as plasticizers.
² e.g., LUDOX (W. R. Grace & Co., Columbia, MD), NYACOL (Nyacol nano Technologies, Inc., Ashland, MA), or CAB-O-SIL (Cabot Corp., Boston, MA)

In some embodiments, the silica/biopolymer composite material may be prepared using alkoxysilanes that produce an alcohol as a byproduct of hydrolysis, such as tetramethoxysilane (TMOS) (which produces methanol as a byproduct), tetraethoxysilane (TEOS) (which produces ethanol as a byproduct) tetrakis(2-hydroxy ethyl)orthosilicate (THEOS) (which produces ethylene glycol as a byproduct), as well as sodium silicate. Composites of the aforementioned silica materials with carbohydrates, e.g., polysaccharides, are also suitable for use as the silica component. In other embodiments, the silica component can include, for example, silica nanoparticles such as LUDOX (7 nm, 12 nm, or 22 nm particles with sodium ion as a counter ion bonded to the silica surface; W.R. Grace & Co.; Columbia, Md.), NEXSIL 85 and 125 (silica nanoparticles with sodium ion as a counterion; Nyacol Nano Technologies; Ashland, Mass.), NEXSIL 20K, 85K and 125K (20 nm, 50 nm, or 85 nm) (silica nanoparticles with potassium ion as a counterion; Nyacol Nano Technologies; Ashland, Mass.), and NEXSIL 20A, 45Z, 85A, 125A (20 nm, 35 nm, 50 nm, or 85 nm) (silica nanoparticles with no counterion; the surface charge is slightly negative; Nyacol Nano Technologies; Ashland, Mass.). In still other embodiments, a preferred silica precursor can include methyltriethoxysilane (MTMOS).

The composites and methods described herein are not limited to any particular silica precursor; numerous silica precursors have been and can be used to form silica gels (see, e.g., Avnir et al. J. Mat. Chem. 2006; 16:1013-1030 for review) that can be suitable for use in the composites and methods described herein.

The biocompatible polymer can include any of the proteins that compose the principal structural proteins of the body, such as, for example, collagens, elastins, keratin, actin, and/or myosin. In some embodiments, the biocompatible polymer includes a collagen.

In embodiments in which the biocompatible polymer includes collagen, the collagen may include any type, or any combinations of types, of collagen including, for example, type I (the most abundant form in the human body), type II, type III, type IV, type V, type VI, type VII type, VIII, type IX, type X, type XI, type XII, type XIII, type XIV, type XV, type XVI, type XVII, type XVIII, type XIX, type XX, type XXI, type XXII, type XIII, type XIV, type XXV, type XXVI, type XXVII, type XXVIII, or type XXIX collagen. The particular type of collagen selected may influence the mechanical properties and/or size of fibrils formed by the collagen. In certain embodiments, type I collagen may be preferred. Collagen may be porcine collagen, but may be collagen from other animal sources, including marine sources, or synthetic collagen. In certain embodiments, natural or recombinant human collagen may be used.

Alternative biocompatible polymers include cellulose or a derivative thereof, such as methyl cellulose; a polypeptide such as, for example, elastin, keratin, actin, or myosin; a polysaccharide such as, for example, cellulose or chitin; an amyloid fiber, a glycosaminoglycan; a proteoglycan; or any combination of biocompatible polymers.

Exemplary silica/biopolymer (e.g., collagen) weight ratios include, for example, 40:1, 35:1, 33:1, 30:1, 25:1, 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:25, 1:30, 1:33, 1:35, and 1:40. Generally, increasing the silica content tends to increase the strength of the silica/biopolymer composite. Generally, increasing the biopolymer content tends to increase the optical quality and/or biocompatibility of the silica/biopolymer composite. For example, a composite having a silica:biopolymer weight ratio of about 3:1 produces fibrils that are similar in diameter to natural fibrils of the cornea. Composites possessing a higher silica content—e.g., a silica:biopolymer weight ratio of about 9:1—can produce fibrils with a larger diameter.

Dehydrating the gel can include incubating the gel in relative humidity of no more than about 80% such as, for example, a relative humidity of no more than 75%, no more than 72%, no more than 70%, no more than 65%, no more than 60%, no more than 55%, no more than 50%, no more than 45%, no more than 40%, no more than 35%, no more than 30%, no more than 25%, no more than 20%, no more than 15%, no more than 10%, no more than 5%, no more than 4%, no more than 3%, no more than 2%, or no more than 1%. In one particular embodiment, dehydrating the gel includes incubating the gel in an atmosphere of 72% relative humidity.

In some embodiments, dehydrating the gel can include incubating the gel at a maximum temperature of no more than 60° C. such as, for example, no more than 50° C., no more than 45° C., no more than 40° C., no more than 35° C., no more than 30° C., no more than 25° C., or no more than 20° C. Dehydrating the gel can include incubating the gel at a minimum temperature of at least 5° C. such as, for example, at least 10° C., at least 15° C., at least 20° C., at least 25° C., at least 30° C., or at least 35° C.

In some embodiments, the temperature at which the gel is dehydrated can fall within a range having endpoints defined by any maximum temperature provided above in combination with any minimum temperature that is less than the maximum temperature. In one particular embodiment, the gel may be dehydrated by incubating the gel at a temperature of from 15° C. to 40° C.

In some embodiments, the gel can be at least partially dehydrated by drying sequentially in solutions with increasing concentrations of an alcohol such as, for example, ethanol. In one such embodiment, the gel may be at least partially dehydrated by sequentially incubating the gel in solutions of 20%, 40%, 60%, 80%, 95%, and finally 100% ethanol. Prior to critical point drying, the gels can be punched using a trephine to produce circular discs of various sizes. These discs can then be placed on a hemispherical polydimethylsiloxan (PDMS) mold and uploaded into a CO₂ critical point dryer. The gels will take the shape of the mold during the drying process. Critical point drying is a dehydration method that improves the mechanical properties of the material while maintaining the original microstructure of the gel.

In some embodiments, the silica precursor and the biocompatible polymer are combined in a solution having a pH of no less than 5. In some embodiments, the silica precursor and the biocompatible polymer are combined in a solution having a pH of no more than 10.

In some embodiments, the pH may be controlled by controlling the amount and/or molarity of acetic acid in the silica precursor solution used to prepare the gel. In such embodiments, a biocompatible polymer/silica solution—as described in Example 1, below—is mixed to a final pH above which biocompatible is stable, which induces fibril formation. With this method, one often performs the mixing on ice to slow down fibril formation, which allows one more time to de-aerate the mixture prior to curing. De-aeration can be accomplished by, for example, centrifuging at 1000 rpm for 10 minutes.

In other embodiments, one can induce fibril formation with ammonia vapors after mixing the biocompatible polymer and the silica precursor and de-aerating without introducing another solvent and more air bubbles. This allows one more time to de-aerate and pour gel into molds while the gel is still in a liquid state. The biocompatible polymer/silica mixture may be placed in an enclosed chamber that holds ammonium hydroxide (pH 8-10). The biocompatible polymer/silica solution's pH increases due to the ammonia vapors, which induces fibril formation. Once the fibril formation is completed, the gel is removed from the chamber.

In still other embodiments, the silica precursor may be added after the biocompatible polymer forms fibrils. As in the methods described immediately above, the molarity and volume of acid (e.g., acetic acid or hydrochloric acid) can modulate the pH of the dilution between a pH of 3 and a pH of 12.

In another aspect, this disclosure also describes a method that generally includes combining a silica precursor with a biocompatible polymer solution under conditions effective for the silica precursor and biocompatible polymer in the solution to form a gel, wherein the solution contains biopolymer at a concentration of at least 5 mg/mL.

The silica precursor and biopolymer used to prepare a biocompatible composite in this manner are the same as those described above.

The concentration of biopolymer in the biopolymer solution can be at least 5 mg/mL such as, for example, at least 10 mg/mL, at least 15 mg/mL, at least 20 mg/mL, at least 25 mg/mL, at least 30 mg/mL, at least 35 mg/mL, at least 40 mg/mL, at least 45 mg/mL, at least 50 mg/mL, at least 60 mg/mL, at least 75 mg/mL, at least 100 mg/mL, at least 125 mg/mL, at least 150 mg/mL, at least 175 mg/mL, at least 200 mg/mL, at least 225 mg/mL, at least 250 mg/mL, at least 275 mg/mL, at least 300 mg/mL, at least 325 mg/mL, at least 350 mg/mL, at least 375 mg/mL, at least 400 mg/mL, at least 450 mg/mL, or at least 500 mg/mL. In one particular embodiment, the biopolymer solution has a concentration of biopolymer that is at least 50 mg/mL. In another particular embodiment, the biopolymer solution has a concentration of biopolymer that is at least 150 mg/mL.

In some cases, the method can include concentrating the biopolymer solution from a more dilute solution of the biopolymer such as, for example, by dialysis.

In the preceding description, particular embodiments may be described in isolation for clarity. Unless otherwise expressly specified that the features of a particular embodiment are incompatible with the features of another embodiment, certain embodiments can include a combination of compatible features described herein in connection with one or more embodiments.

For any method disclosed herein that includes discrete steps, the steps may be conducted in any feasible order. And, as appropriate, any combination of two or more steps may be conducted simultaneously.

The present invention is illustrated by the following examples. It is to be understood that the particular examples, materials, amounts, and procedures are to be interpreted broadly in accordance with the scope and spirit of the invention as set forth herein.

### EXAMPLES

### Example 1

### Composite Preparation

For all tests and applications, the fabrication of the composite material followed a standard procedure. Soluble type I bovine collagen (Organogenesis Collagen, Canton, Mass.) was mixed with the diluted aminopropyl triethyl silane (APTS, Sigma-Aldrich; St. Louis, Mo.) at a 10:3 volume ratio for 10 minutes with a magnetic stir bar. To prevent premature gelation, the mixing vial was submerged in an ice bath.

Aminopropyl triethyl silane was mixed with acetic acid in a bath sonicator at 4° C. The silica to collagen ratio was modulated with the volume of acetic acid used in the APTS dilution. Also, the molarity of the acetic acid was varied to adjust the pH of the composites. In this study, we investigated two compositions: one having a silica:collagen weight ratio of 3:1; the other having a silica:collagen weight ratio of 9:1. The composition having the 3:1 silica:collagen ratio was prepared at a pH of 6; The composition having the 9:1 silica:collagen weight ratio was prepared at a pH of 8.

Immediately after mixing, the gels were de-aerated by centrifugation and poured into polydimethylsiloxaine (PDMS) negative molds. For manufacturing of the ex-vivo implants, the gel was allowed to cure in a hemispherical mold, with curvature similar to that of the cornea, and then punched with a corneal trephine to the appropriate diameter. Conventional methods of composite manufacturing involved formation of the composite under fully hydrated conditions (100% relative humidity). Composites manufactured in this method will be referred to as hydrogels. Additional studies were performed on composites that were dehydrated (cured at 72% relative humidity for 24 hours at room temperature) which are specified as ‘xerogels’. After complete formation and dehydration of the material, the samples were rehydrated in a phosphate buffered saline (PBS, Sigma-Aldrich, St. Louis, Mo.) solution. The rehydrated samples were kept in 100% relative humidity conditions prior to testing. Because all xerogels were rehydrated prior to testing, rehydrated xerogels will be referred to as simply xerogels.

### Example 2

### Optical Measurements

The transmittance, T, defined as the ratio of transmitted light intensity to incident light intensity, of each composition was measured in a 96 well plate by using a spectrophotometer (SpecroMax Plus, Molecular Devices; Sunnyvale, Calif.). A 50 μL volume of mixed gel was pipetted into each well. The gel was allowed to cure inside the well at 100% humidity. Each well was hydrated with 50 microliters of phosphate-buffered solution (PBS). Twelve wells were filled with 50 μL of PBS and used as blanks. The fraction of transmittance (ratio of the amount of light that passed through the sample to the amount of light that passed through the blank) was calculated for wavelengths of 300 to 800 nm in 10 nm intervals. With knowledge of the transmittance and thickness of a sample, d, a material constant α_{c} for each composition was calculated from equation 1.

Log^{T-1}=α_{c}d (1)

The material constant is an intrinsic material property. With a known α_{c} and by using the same material thickness (d), transmittance of two different compositions could be directly compared. Transmittance can be considered an intrinsic property for a fixed thickness, d.

Refractive indices of the materials were measured with an Abbe refractometer (Zeiss) at 25° C. The composites were individually immersed in saturated sucrose solutions. The solution that contained a sample was diluted dropwise with distilled water, until the sample became more transparent. An aliquot of the sucrose solution was removed and its refractive index was measured. The solution was then diluted again by a few drops of distilled water, and the refractive index was re-measured. Changes in refractive index between dilutions were approximately 0.004. This procedure was repeated until the sample became more opaque, and the refractive index of this solution was recorded.

### Example 3

### Suture Pullout Measurements

The ability of the composite material to hold a suture was estimated by using a low force biaxial tester (Instron; Norwood, Mass.) to measure tensile strength of the sample. An 8 mm-diameter corneal trephine was used to punch out disc-shaped test implants. One half of the disc was clamped in a spring-loaded grip. A 10-0 nylon suture was placed 2 mm from the edge of the opposite half of the implant, and the two free ends of the suture were placed in the other grip. All implants were tested in a PBS bath to prevent dehydration. The grip holding the suture was displaced at a rate of 0.1 mm/second relative to the opposing grip until complete the suture pulled through the implant. Rupture strength was defined as the force at failure divided by the thickness of the disc.

### Example 4

### Scanning Electron Microscopy

The morphology and microstructure of the composite materials was assessed by scanning electron microscopy (SEM). Implants were fixed in formaldehyde (4 wt % in water, Sigma-Aldrich; St. Louis, Mo.), dried sequentially in 20%, 40%, 60%, 80%, 95%, and finally 100% ethanol solutions (Pharmco-AAPER), then transferred to a Tousimis samdri-780A CO₂ critical point dryer (Tousimis; Rockville, Md.). Samples (5 mm discs) were broken in half and mounted on carbon tape stubs, one half flat against the stub, the other perpendicular for cross-sectional imaging. The dried specimens were then coated in 5 nm of Pt and imaged on a JEOL 6700F SEM with a 5 kV beam at 10 μA.

### Example 5

### Ex Vivo Biocompatibility in a Rabbit Model

Preliminary biocompatibility of corneal re-epithelialization of the 9:1 hydrogel was studied in a rabbit cornea organ culture model similar to a method described by Evans et al. (Biomaterials. 2002; 23(5):1359-67). Five New Zealand white rabbits were euthanized with an overdose of intravenous sodium pentobarbital immediately before operating on both corneas. All the procedures involving these animals adhered to the ARVO Statement for Use of Animals in Ophthalmic and Vision Research and were approved by the Mayo Clinic Institutional Animal Use and Care Committee. Under sterile conditions, the center of the cornea was marked and the anterior stroma was incised with a guarded diamond blade to a depth of 200 μm. A 9 mm-diameter circular lamellar pocket was created at a depth of 200 μm from the anterior surface. The anterior lamella was excised centrally to create a keratectomy approximately 5 mm in diameter.

After surgical preparation of the cornea for implantation, the eye was enucleated and the anterior segment was excised, including removal of the lens and iris. The anterior segment had an approximately 3 mm rim of sclera and was mounted to a modified Petri dish (Brunette et al. Invest. Ophth. & Visual Sci. 1989; 30(8):1813-1822) designed for rabbit anterior segment organ culture (FIG. 1). The corneoscleral rim was clamped with a locking ring and culture medium was infused to fill and maintain the anterior chamber. The culture medium was Dulbecco's Modified Eagles Medium/Ham's F-12 with 20 mM L-Glutamine (Sigma-Aldrich, St. Louis, Mo.) with a 1:100 dilution of Antibiotic/Antimycotic Suspension (Sigma-Aldrich; pencillin G 100 units/mL, streptomycin 100 μg/mL, amphotericin B 0.25 μg/mL) and a 1:100 dilution of Insulin-Transferrin-Selenium (Gibco, Carlsbad, Calif.). From a sterile film of the composite, an 8.2 mm button was created by punching with a corneal trephine. The button was then implanted into 8 corneas with the periphery of the implant enveloped in the lamellar pocket and the center of the implant exposed because of the keratectomy. The dimensions of the keratectomy, i.e., the de-epithelialized region over the implant, were measured by staining the region with sodium fluorescein and examining with a blue light (FIG. 2). Culture medium was placed externally to just cover the center of the cornea and implant.

Two control eyes received an identical surgical procedure but without implantation of a bio-composite. In all cases, the epithelial defect was measured daily until closure, and time to closure was recorded. Culture medium was changed daily. Anterior segments were cultured for 7-18 days, after which the corneoscleral rims were fixed for examination by light microscopy.

### Statistical Analysis

All mean values in this paper are presented with the standard deviation.

### EXEMPLARY EMBODIMENTS

### Embodiment 1

A method comprising:

combining a silica precursor and a biopolymer under conditions effective for the silica precursor and the biocompatible polymer to form a gel;

at least partially dehydrating the gel; and

rehydrating the gel.

### Embodiment 2

The method of Embodiment 1 wherein the biopolymer comprises Type I collagen.

### Embodiment 3

The method of any preceding Embodiment wherein the silica precursor and the biopolymer are provided in amounts to produce a gel comprising a silica:biopolymer ratio of no more than 9:1.

### Embodiment 4

The method of Embodiment 3 wherein the silica:biopolymer ratio is no more than 3:1.

### Embodiment 5

The method of any preceding Embodiment wherein dehydrating the gel comprises incubating the gel in relative humidity of no more than 72%.

### Embodiment 6

The method of any preceding Embodiment wherein dehydrating the gel comprises incubating the gel for at least 24 hours.

### Embodiment 7

The method of any preceding Embodiment wherein rehydrating the gel comprises incubating the at least partially dehydrated gel in a buffer.

### Embodiment 8

The method of any preceding Embodiment further comprising storing the rehydrated gel in 100% relative humidity conditions.

### Embodiment 9

The method of any preceding Embodiment wherein the silica precursor and the biopolymer are combined in a solution having a pH of no less than 5.

### Embodiment 10

The method of any preceding Embodiment wherein the silica precursor and the biopolymer are combined in a solution having a pH of no more than 10.

### Embodiment 11

A corneal implant prepared by the method of any one of Embodiments 1-10.

### Embodiment 12

A method comprising:
combining a silica precursor, a biopolymer, and aqueous nano-crystalline cellulose under conditions effective for the silica precursor and the biopolymer to form a gel.

### Embodiment 13

The method of Embodiment 12 wherein the biopolymer comprises Type I collagen.

### Embodiment 14

The method of Embodiment 12 or Embodiment 13 wherein the silica precursor and the biopolymer are provided in amounts to produce a gel comprising a silica:biopolymer ratio of no more than 9:1.

### Embodiment 15

The method of Embodiment 14 wherein the silica:biopolymer ratio is no more than 3:1.

### Embodiment 16

The method of any one of Embodiments 12-15 wherein the silica precursor and the biocompatible polymer are combined in a solution having a pH of no less than 5.

### Embodiment 17

The method of any one of Embodiments 12-16 wherein the silica precursor and the biopolymer are combined in a solution having a pH of no more than 10.

### Embodiment 18

A corneal implant prepared by the method of any one of Embodiments 12-17.

### Embodiment 19

A method comprising:
combining a silica precursor and a biopolymer solution under conditions effective for the silica precursor and the biopolymer in the solution to form a gel, wherein the biopolymer solution comprises the biopolymer at a concentration of at least 5 mg/mL.

### Embodiment 20

The method of Embodiment 19 further comprising concentrating a solution of the biopolymer.

### Embodiment 21

The method of Embodiment 19 or Embodiment 20 wherein the biopolymer comprises Type I collagen.

### Embodiment 22

The method of any one of Embodiments 19-21 wherein the silica precursor and the biopolymer are provided in amounts to produce a gel comprising a silica:biopolymer ratio of no more than 9:1.

### Embodiment 23

The method of Embodiment 23 wherein the silica:biopolymer ratio is no more than 3:1.

### Embodiment 24

The method of any one of Embodiments 19-23 wherein the silica precursor and the biocompatible polymer are combined in a solution having a pH of no less than 5.

### Embodiment 25

The method of any one of Embodiments 19-24 wherein the silica precursor and the biopolymer are combined in a solution having a pH of no more than 10.

### Embodiment 26

A corneal implant prepared by the method of any one of Embodiments 19-25.

The complete disclosure of all patents, patent applications, and publications, and electronically available material cited herein are incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

The term “and/or” means one or all of the listed elements or a combination of any two or more of the listed elements; the terms “comprises” and variations thereof do not have a limiting meaning where these terms appear in the description and claims; unless otherwise specified, “a,” “an,” “the,” and “at least one” are used interchangeably and mean one or more than one; and the recitations of numerical ranges by endpoints include all numbers subsumed within that range (e.g., 1 to 5 includes 1, 1.5, 2, 2.75, 3, 3.80, 4, 5, etc.).

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

What is claimed is:

1. A method comprising:
combining a silica precursor and a biopolymer under conditions effective for the silica precursor and the biopolymer to form a gel;
at least partially dehydrating the gel; and
rehydrating the gel.

2. The method of claim 1 wherein the biopolymer comprises Type I collagen.

3. The method of claim 1 wherein the silica precursor and the biopolymer are provided in amounts to produce a gel comprising a silica:biopolymer ratio of no more than 9:1.

4. The method of claim 3 wherein the silica:biopolymer ratio is no more than 3:1.

5. The method of claim 1 wherein dehydrating the gel comprises incubating the gel in relative humidity of no more than 72%.

6. The method of claim 1 wherein dehydrating the gel comprises incubating the gel for at least 24 hours.

7. The method of claim 1 wherein rehydrating the gel comprises incubating the at least partially dehydrated gel in a buffer.

8. The method of claim 1 further comprising storing the rehydrated gel in 100% relative humidity conditions.

9. The method of claim 1 wherein the silica precursor and the biopolymer are combined in a solution having a pH of no less than 5.

10. The method of claim 1 wherein the silica precursor and the biopolymer are combined in a solution having a pH of no more than 10.

11. A corneal implant prepared by the method of claim 1.

12. A method comprising:
combining a silica precursor, a biopolymer, and aqueous nano-crystalline cellulose under conditions effective for the silica precursor and the biopolymer to form a gel.

13. The method of claim 12 wherein the biopolymer comprises Type I collagen.

14. The method of claim 12 wherein the silica precursor and the biopolymer are provided in amounts to produce a gel comprising a silica:biopolymer ratio of no more than 9:1.

15. The method of claim 14 wherein the silica:biopolymer ratio is no more than 3:1.

16. The method of claim 12 wherein the silica precursor and the biocompatible polymer are combined in a solution having a pH of no less than 5.

17. The method of claim 12 wherein the silica precursor and the biopolymer are combined in a solution having a pH of no more than 10.

18. A corneal implant prepared by the method of claim 12.

19. A method comprising:
combining a silica precursor and a biopolymer solution under conditions effective for the silica precursor and the biopolymer in the solution to form a gel, wherein the biopolymer solution comprises the biopolymer at a concentration of at least 5 mg/mL.

20. The method of claim 19 further comprising concentrating a solution of the biopolymer to achieve a biopolymer concentration of at least 5 mg/mL.

21. The method of claim 19 wherein the biopolymer comprises Type I collagen.

22. The method of claim 19 wherein the silica precursor and the biopolymer are provided in amounts to produce a gel comprising a silica:biopolymer ratio of no more than 9:1.

23. The method of claim 22 wherein the silica:biopolymer ratio is no more than 3:1.

24. The method of claim 19 wherein the silica precursor and the biocompatible polymer are combined in a solution having a pH of no less than 5.

25. The method of claim 19 wherein the silica precursor and the biopolymer are combined in a solution having a pH of no more than 10.

26. A corneal implant prepared by the method of claim 19.
